(19) United States
(12) Patent Application Publication
(10) Pub. No.: US 2022/0146452 A1
(43) Pub. Date: May 12, 2022

(54) DETECTOR, DETECTION METHOD, AND PROGRAM

(71) Applicant: Murata Manufacturing Co., Ltd., Nagaokakyo-shi (JP)

(72) Inventors: Tsuyoshi OKINO, Nagaokakyo-shi (JP); Kohei TAKAHASHI, Nagaokakyo-shi (JP); Shota USHIBA, Nagaokakyo-shi (JP)

(21) Appl. No.: 17/580,785

(22) Filed: Jan. 21, 2022

Related U.S. Application Data
(63) Continuation of application No. PCT/JP2020/026837, filed on Jul. 9, 2020.

(30) Foreign Application Priority Data
Aug. 1, 2019 (JP) 2019-142490
Apr. 7, 2020 (JP) 2020-069205

(51) Int. Cl. G01N 27/414

(52) U.S. Cl. CPC G01N 27/4146

(57) ABSTRACT

A detector detects a target using a sensor and includes a measurement circuit to measure a signal from the sensor and a computation circuit to separate a signal measured by the measurement circuit into a variation component of the sensor and a response component of the sensor. The computation circuit performs analysis using a state space model including a state equation specified by time-series information of the variation component of the sensor and an observation equation specified by separation between the variation component of the sensor and the response component of the sensor.

### CROSS REFERENCE TO RELATED APPLICATIONS

This application claims the benefit of priority to Japanese Patent Application No. 2019-142490 filed on Aug. 1, 2019, Japanese Patent Application No. 2020-069205 filed on Apr. 7, 2020 and is a Continuation Application of PCT Application No. PCT/JP2020/026837 filed on Jul. 9, 2020. The entire contents of each application are hereby incorporated herein by reference.

### BACKGROUND OF THE INVENTION

### 1. Field of the Invention

The present invention relates to a detector including one or more sensors, a detection method, and a non-transitory computer-readable medium storing a program.

### 2. Description of the Related Art

For the detection of chemical or biochemical compounds, various sensors, such as a potentiometric sensor (e.g., Japanese Unexamined Patent Application Publication No. 2016-121992) and a glucose sensor that continuously measuring glucose values (e.g., Japanese Unexamined Patent Application Publication No. 2018-532440) have been developed. A problem common in these sensors is that the response component of a sensor is not output as a signal from the sensor and a signal obtained by superimposing the variation component (drift component) of the sensor upon the response component is output as a signal from the sensor.

Accordingly, in Japanese Unexamined Patent Application Publication No. 2016-121992, an additional electrode is provided for the compensation of a drift component of a reference voltage superimposed on an ion sensor that is a potentiometric sensor. In Japanese Unexamined Patent Application Publication No. 2018-532440, a value in a steady state is measured in advance for the calibration of a drift component superimposed on a glucose sensor that is an analyte concentration sensor in a biological system, and calibration is performed using the measured value. As a method of calibrating a drift component included in a signal from a sensor, a method is also known of approximately calibrating a signal from a sensor on condition that a drift component linearly changes.

However, in the method approximately performed on a condition that a drift component linearly changes, the accuracy of approximation decreases when a drift component included in a signal from a sensor is a nonlinear component. This may lead to the reduction in accuracy of detecting a compensated target. In Japanese Unexamined Patent Application Publication No. 2016-121992, an additional piece of hardware such as an electrode is needed for the compensation of a drift component of a reference voltage superimposed on an ion sensor that is a potentiometric sensor. This leads to a complicated configuration of a device and an increase in the cost of manufacturing the device. In Japanese Unexamined Patent Application Publication No. 2018-532440, detection needs to be performed after a detection target has gone into a steady state because a value in a steady state is measured in advance and calibration is performed using the measured value. This results in a problem that it takes time to complete detection.

### SUMMARY OF THE INVENTION

Preferred embodiments of the present invention provide detectors, detection methods, and non-transitory computer-readable media storing programs, each of which enabling a target to be accurately detected without the need to add another piece of hardware and the need to wait until the target goes into a steady state.

A detector according to a preferred embodiment of the present invention detects a target using a sensor. The detector includes a measurement circuit to measure a signal from the sensor and a computation circuit to separate a signal measured by the measurement circuit into a variation component of the sensor and a response component of the sensor. The computation circuit includes a state space model analysis portion to perform analysis using a state space model including a state equation specified by time-series information of a variation component of the sensor and an observation equation specified by separation between a variation component of the sensor and a response component of the sensor and a parameter determination portion configured to determine a parameter included in the state space model used by the state space model analysis portion. The computation circuit obtains a target corresponding to a response component using a parameter determined by the parameter determination portion.

According to preferred embodiments of the present invention, a target is able to be accurately detected without adding another piece of hardware and waiting until the target goes into a steady state because a computation circuit performs analysis using a state space model including a state equation specified by the time-series information of the variation component of a sensor and an observation equation specified by the separation between the variation component of the sensor and the response component of the sensor.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic diagram illustrating a configuration of a detector according to a first preferred embodiment of the present invention.

FIG. 2 is a schematic diagram illustrating a configuration of a computation circuit according to the first preferred embodiment of the present invention.

FIG. 3 is a flowchart of a learning phase in the first preferred embodiment of the present invention.

FIGS. 4A and 4B are graphs representing a change in measurement value in a learning phase in the first preferred embodiment of the present invention.

FIGS. 5A and 5B are graphs representing the change in measurement value in a learning phase in the first preferred embodiment of the present invention.

FIGS. 6A and 6B are diagrams illustrating parameters of a response model estimated in a learning phase in the first preferred embodiment of the present invention.

FIG. 7 is a flowchart of a prediction phase in the first preferred embodiment of the present invention.

FIGS. 8A and 8B are graphs representing a change in measurement value in a prediction phase in the first preferred embodiment of the present invention.

FIG. 9 is a diagram illustrating a concentration of a protein solution calculated in a prediction phase in the first preferred embodiment of the present invention.

FIG. 10 is a block diagram illustrating a configuration of a computer according to the first preferred embodiment of the present invention.

FIG. 11 is a schematic diagram illustrating a configuration of a detector according to a second preferred embodiment of the present invention.

FIG. 12 is a flowchart of a learning phase in the second preferred embodiment of the present invention.

FIG. 13 is a flowchart of a prediction phase in the second preferred embodiment of the present invention.

### DETAILED DESCRIPTION OF THE PREFERRED EMBODIMENTS

Detectors according to preferred embodiments of the present invention will be described in detail below with reference to the accompanying drawings. In the drawings, the same reference numeral is used to represent the same elements or portions or the corresponding elements or portions.

### First Preferred Embodiment

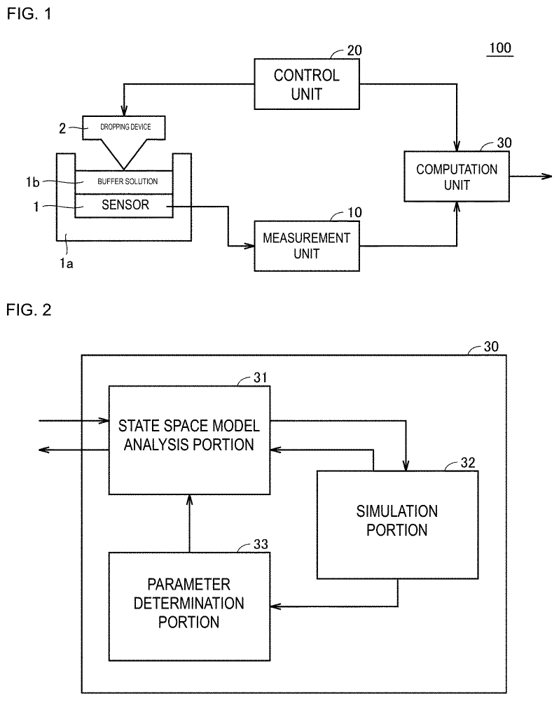

A detector according to a first preferred embodiment of the present invention will be described below with reference to the accompanying drawings. FIG. 1 is a schematic diagram illustrating the configuration of a detector according to the first preferred embodiment. A detector 100 illustrated in FIG. 1 detects the concentration of a protein solution that is a detection target using, for example, a graphene FET sensor. A graphene FET sensor is an FET sensor including a graphene film on a base. A graphene film shows a significant change in electrical characteristics in response to the binding, adsorption, or proximity of atoms or molecules on the surface of the film. Accordingly, a graphene FET sensor including the graphene film used as an ion sensor, an enzyme sensor, a DNA sensor, an antigen-antibody sensor, a protein sensor, a breath sensor, a gas sensor, and other sensors, for example.

A graphene FET sensor (hereinafter also referred to as sensor) 1 is provided in a casing 1a and includes an upper surface filled with a buffer solution 1b. As the buffer solution 1b, for example, PBS (phosphate buffered salts) is used. A protein solution, which is a detection target, is dropped in the buffer solution 1b from a dropping device 2. The dropping device 2 is, for example, a micropipette. The detector 100 detects the concentration of a protein solution dropped from the dropping device 2 as a target while continuously monitoring current values output from the sensor 1. The concentration of a protein solution is a detection target in this example, but the concentration of an ion, an enzyme, a DNA, an antigen, or an antibody, for example, may be a detection target.

The sensor 1 is a graphene FET sensor in the present preferred embodiment, but may be another type of sensor such as, for example, an Si-FET sensor, a carbon nanotube FET, a silicon nanowire FET, or a diamond FET. The detector 100 is applicable to a temperature sensor, a gas sensor, or an inertial sensor in which a variation component (drift component) is generated.

The detector 100 includes the sensor 1, a measurement circuit 10, a controller 20, and a computation circuit 30. Although the detector 100 includes the sensor 1 in the present preferred embodiment, a sensor may be disposed outside a detector and the detector may detect a target based on a signal from the sensor. In the detector 100, the controller 20 controls the dropping device 2 to drop a protein solution, which is a detection target, in the buffer solution 1b. However, the controller 20 does not necessarily have to control the dropping device 2 and the dropping may be manually performed. A detector does not necessarily have to include a dropping device.

The measurement circuit 10 measures signals from the sensor 1 to continuously monitor current values. The measurement circuit 10 has a configuration based on the configuration of the sensor 1. The measurement circuit 10 includes an ammeter when measuring the current value of the sensor 1 and includes a voltmeter when measuring the voltage value of the sensor 1.

The controller 20 controls the operation of the entire of the detector 100, and controls the operations of, for example, the sensor 1, the dropping device 2, the measurement circuit 10, and the computation circuit 30. FIG. 1 illustrates an exemplary case where the controller 20 controls the dropping of the dropping device 2 and the computation of the computation circuit 30. Specifically, the controller 20 can control the dropping timing and the amount of a protein solution from the dropping device 2 and output information about them to the computation circuit 30. When the dropping device 2 drops a protein solution of known concentration, the controller 20 can output information about the known concentration to the computation circuit 30.

The controller 20 can also control a computation phase in the computation circuit 30. The computation circuit 30 can separate a current value (signal) measured by the measurement circuit 10 into the variation component of the sensor 1 and the response component of the sensor 1 using a state space model. Accordingly, the computation circuit 30 includes a learning phase (first computation phase) in which the parameter of a state space model to be described below is determined and a prediction phase (second computation phase) in which the concentration (target) of a protein solution is obtained based on the determined parameter. The controller 20 controls the computation circuit 30 to cause the computation circuit 30 to compute in the learning phase or the prediction phase.

FIG. 2 is a schematic diagram illustrating the configuration of the computation circuit 30 according to the first preferred embodiment. The computation circuit 30 includes a state space model analysis portion 31, a simulation portion 32, and a parameter determination portion 33. The state space model analysis portion 31 performs analysis using a state space model including a state equation specified by the time-series information of the variation component of the sensor 1 and an observation equation specified by the separation between the variation component of the sensor 1 and the response component of the sensor 1. In the present preferred embodiment, the variation component of the sensor 1 is handled as “state” in a state space model and a result of an actually performed “observation” is handled as a signal from the sensor 1.

Specifically, in a state space model, a signal from the sensor 1 which includes a variation component (drift component) can be expressed using two equations, a state equation and an observation equation.

State equation: xₜ=G(xₜ₋₁, wₜ)

Observation equation: yₜ=F(xₜ, qₜ, vₜ)

In these equations, xₜ represents the variation component (drift component) of the sensor 1, yₜ represents a signal from the sensor 1 (a current value measured by the measurement circuit 10), qₜ represents a response model representing the relationship between the concentration of a protein solution that is a detection target and the response quantity of the sensor 1, wₜ represents system noise, and vₜ represents observation noise. Each of the variables (e.g., xₜ and wₜ) in the equations may be a vector quantity. For example, by using xₜ=(xₜ, xₜ₋₁) and xₜ₋₁=(xₜ₋₁, xₜ₋₂) in the above state equations, a state up to two previous time points can be handled.

The system noise wₜ and the observation noise vₜ do not necessarily have to have a normal distribution and may have another distribution such as, for example, a Cauchy distribution or a t distribution. The parameters of distributions of the system noise wₜ and the observation noise vₜ and the parameter of the response model qₜ can be obtained in a collective manner by mathematical calculation, and do not necessarily have to have respective fixed values in advance. For the addition of constraints in mathematical calculation, the parameter of the distribution of each noise and the parameter of the response model qₜ may be determined in advance as distributions.

Even if a function for the variation component of the sensor 1 in a state space model is not known in advance, it can be expressed as a state equation specified by the time-series information of the variation component of the sensor 1. Specifically, a state equation and an observation equation are specified as follows in the present preferred embodiment.

State equation: xₜ−xₜ₋₁=xₜ₋₁−xₜ₋₂+wₜ wₜ˜N(0, σ_{w})

Observation equation: yₜ=xₜ+qₜ+vₜ vₜ˜N(0, σᵥ)

The above state equation is a second-order difference model and can express a gradual time-series change xₜ. Since the variation component of the sensor 1 is considered to gradually change, a second-order difference model is more adequate for the state equation. The observation equation models the fact that a result of the addition of a gradual variation component, a response component to protein, and observation noise is obtained as a signal.

As the response model qₜ, any model such as, for example, a nonlinear model can be used. Specifically, the response model qₜ is specified by the following equation in the present preferred embodiment.

Response model: qₜ=(cₜ/(10^{a}+cₜ))·b

In this equation, cₜ represents the concentration of a protein solution at a time t and a and b represent the parameters of the response model qₜ. The above equation is a Langmuir's adsorption isotherm equation and models the phenomenon in which solutes in solutions are subjected to adsorption on a surface of a solid object. Since a concentration can be detected at the time of adsorption of protein on the sensor 1, the above Langmuir's adsorption isotherm is applied to the response model qₜ in the present preferred embodiment. The response model qₜ is not limited thereto, and may be modeled using other nonlinear functions. The number of parameters of the response model qₜ may be any number.

The state space model analysis portion 31 can obtain the concentration of a protein solution by analyzing the above state space model and separating the variation component (drift component) of the sensor 1 from a signal from the sensor 1 (a current value measured by the measurement circuit 10). However, in order to allow the state space model analysis portion 31 to obtain the concentration of a protein solution from the state space model, the parameter determination portion 33 needs to determine a parameter included in the state space model in advance. In the above state space model, the parameter determination portion 33 needs to determine the parameters a and b of the response model qₜ in advance.

Since the response model qₜ that is a nonlinear function is included in the above state space model, it is difficult for the state space model analysis portion 31 to analytically derive a solution. Accordingly, the simulation portion 32 is provided in the computation circuit 30. The simulation portion 32 derives a solution by performing mathematical calculation by simulation upon the state space model including the response model qₜ that is a nonlinear function. For example, the simulation portion 32 derives a solution by performing mathematical calculation by simulation upon the state space model using the Markov chain Monte Carlo (MCMC) method. In mathematical calculation performed by the simulation portion 32, the Markov chain Monte Carlo method does not necessarily have to be used and another mathematical calculation method may be used. When a nonlinear function is not included in a state space model or when a solution can be analytically derived even in the case of a state space model including a nonlinear function, the simulation portion 32 does not necessarily have to be provided and the computation circuit 30 may perform the analysis.

In the above state space model, the response model qₜ is included in the observation equation. However, the response model qₜ may be included in the state equation. Alternatively, the state equation may be divided into two or more equations, and the response model qₜ may be included in one of these equations.

Next, the learning phase (first computation phase) in the computation phase of the computation circuit 30 will be described. The learning phase is a computation phase in which the parameters a and b of the response model qₜ are determined. Specifically, in the learning phase, the parameters a and b of the response model qₜ are determined based on a signal that is output from the sensor 1 after a protein solution of known concentration has been dropped on the sensor 1.

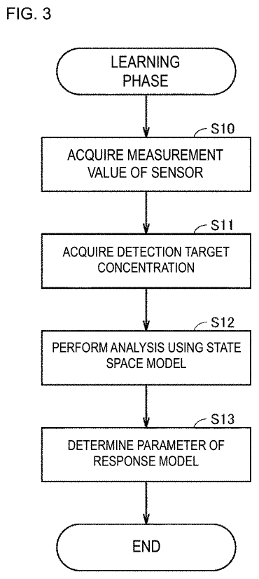

FIG. 3 is a flowchart of the learning phase in the first preferred embodiment. First, the computation circuit 30 acquires from the measurement circuit 10 a measurement value (current value) measured by the sensor 1 (step S10). Subsequently, the computation circuit 30 acquires from the controller 20 a known protein solution concentration (detection target concentration) (step S11). In step S11, the computation circuit 30 does not necessarily have to acquire a known protein solution concentration from the controller and may receive the input of a known protein solution concentration from a user.

The computation circuit 30 causes the state space model analysis portion 31 to perform analysis using the above state space model (step S12). The computation circuit 30 causes the simulation portion 32 to perform mathematical calculation by simulation upon the above state space model to determine the parameters a and b of the response model qₜ (step S13).

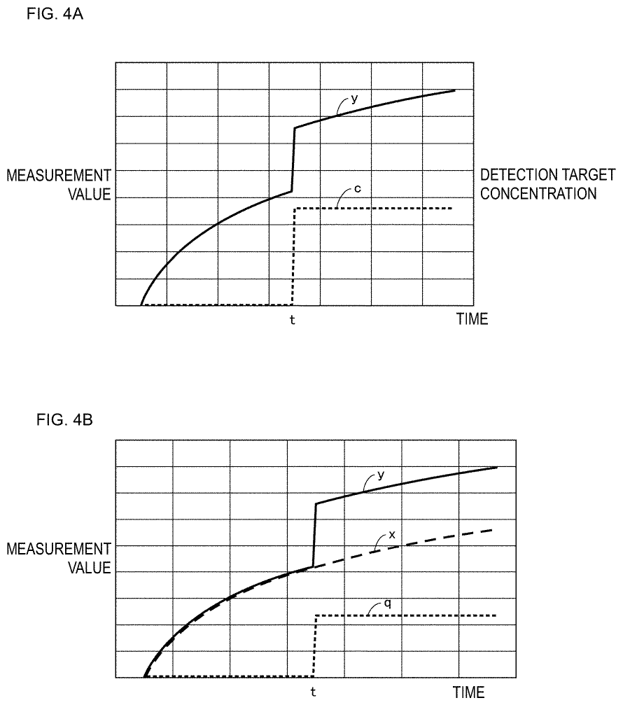

Next, a concrete example of the learning phase will be described. FIGS. 4A and 4B are graphs representing the change in measurement value in the learning phase in the first preferred embodiment. FIG. 4A illustrates changes in a signal (current value measured by the measurement circuit 10) y from the sensor 1 and a protein solution concentration (detection target concentration) c. The vertical axis of y represents measurement value. The vertical axis of c represents detection target concentration. The horizontal axis represents time. FIG. 4B illustrates changes in the signal (current value measured by the measurement circuit 10) y from the sensor 1, a variation component (drift component) x of the sensor 1, and a response component (response model) q of the sensor 1. The vertical axis represents measurement value and the horizontal axis represents time.

For example, the measurement values in FIG. 4 are obtained by continuously monitoring a drain current in a state where a predetermined voltage is applied to the gate electrode and the drain electrode of the sensor 1 in a graphene FET. FIGS. 4A and 4B illustrate a change in measurement value when a protein solution of known concentration is dropped in the buffer solution 1b at a time t.

As illustrated in FIG. 4A, the signal (current value measured by the measurement circuit 10) y from the sensor 1 represented by a solid line nonlinearly changes even before a protein solution of known concentration is dropped, and a variation component (drift component) is superimposed on the signal. When a protein solution of known concentration is dropped at the time t, the signal y from the sensor 1 also changes in accordance with the change in the protein solution concentration (detection target concentration) c represented by a broken line.

The computation circuit 30 can separate the signal y from the sensor 1 into the variation component (drift component) x of the sensor 1 and a response component (response model) q of the sensor 1 as illustrated in FIG. 4B by performing analysis using the above state space model in step S12. In analysis performed using a state space model, the variation component x of the sensor 1 and the response component q of the sensor 1 can be subjected to distribution estimation rather than point estimation. FIG. 4B illustrates the mean value of the variation components x of the sensor 1 obtained by distribution estimation and the mean value of the response components q of the sensor 1 obtained by distribution estimation. By analyzing the variation component of the sensor 1 using a state space model, the variation component of the sensor 1 can be quantitatively determined separately from the response component q of the sensor 1 even if a function for the variation component of the sensor 1 is not known in advance.

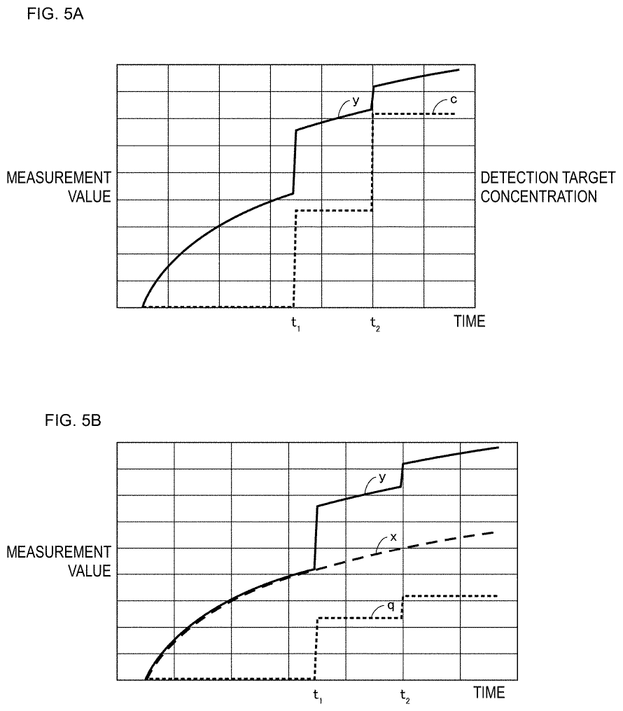

FIGS. 4A and 4B illustrate the change in measurement value when one type of protein solution of known concentration is dropped on the sensor 1. Next, the change in measurement value when two or more types of protein solutions of known concentration are intermittently dropped on the sensor 1 will be described. FIGS. 5A and 5B are graphs representing the change in measurement value in the learning phase in the first preferred embodiment.

FIG. 5A illustrates the changes in the signal (current value measured by the measurement circuit 10) y from the sensor 1 and the protein solution concentration (detection target concentration) c. The vertical axis of y represents measurement value, the vertical axis of c represents detection target concentration, and the horizontal axis represents time. FIG. 5B illustrates the changes in the signal (current value measured by the measurement circuit 10) y from the sensor 1, the variation component (drift component) x of the sensor 1, and the response component (response model) q of the sensor 1. The vertical axis represents measurement value and the horizontal axis represents time.

FIGS. 5A and 5B illustrate changes in measurement value when a first type of protein solution of known concentration is dropped in the buffer solution 1b at a time t₁ and a second type of protein solution of known concentration is dropped in the buffer solution 1b at a time t₂.

As illustrated in FIG. 5A, the signal (current value measured by the measurement circuit 10) y from the sensor 1 represented by a solid line nonlinearly changes even before a protein solution of known concentration is dropped, and a variation component (drift component) is superimposed on the signal. When the first type of protein solution of known concentration is dropped at the time t₁, the signal y from the sensor 1 also changes in accordance with the change in the protein solution concentration (detection target concentration) c represented by a broken line. When the second type of protein solution of known concentration is dropped at the time t₂, the signal y from the sensor 1 also changes in a stepwise manner in accordance with the change in the protein solution concentration (detection target concentration) represented by the broken line.

Even when two or more types of protein solutions of known concentration are intermittently dropped on the sensor 1, the computation circuit 30 can separate the signal y from the sensor 1 into the variation component (drift component) x of the sensor 1 and the response component (response model) q of the sensor 1 as illustrated in FIG. 5B by performing analysis using the above state space model in step S12. FIG. 5B illustrates the mean value of the variation components x of the sensor 1 obtained by distribution estimation and the mean value of the response components q of the sensor 1 obtained by distribution estimation.

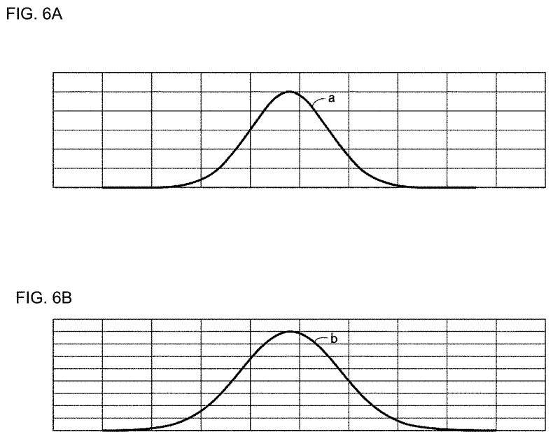

The computation circuit 30 causes the simulation portion 32 and the parameter determination portion 33 to estimate the parameters a and b of the response model qₜ using the MCMC method. FIGS. 6A and 6B are diagrams illustrating the parameters a and b of the response model qₜ estimated in the learning phase in the first preferred embodiment. FIG. 6A illustrates the distribution of the estimated parameter a of the response model qₜ. FIG. 6B illustrates the distribution of the estimated parameter b of the response model qₜ. By estimating the parameters a and b of the response model qₜ, not only the response component (response model) q of the sensor 1 but also the characteristics of the sensor 1 can be evaluated. In FIGS. 6A and 6B, the horizontal axis represents parameter value and the vertical axis represents frequency.

Next, a prediction phase (second computation phase) in the computation phase of the computation circuit 30 will be described. The prediction phase is a computation phase in which an unknown concentration of a protein solution is predicted using results of the parameters a and b of the response model qₜ determined in the learning phase. Specifically, in the prediction phase, a protein solution of unknown concentration is dropped on the sensor 1 and the concentration of the protein solution is obtained based on a signal from the sensor 1.

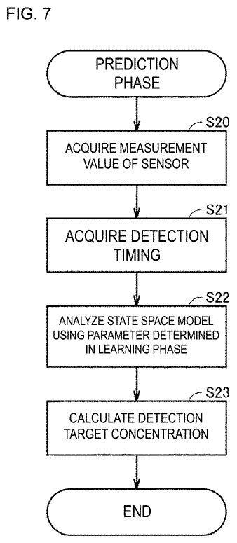

FIG. 7 is a flowchart of the prediction phase in the first preferred embodiment. First, the computation circuit 30 acquires from the measurement circuit 10 a measurement value (current value) measured by the sensor 1 (step S20). Subsequently, the computation circuit 30 acquires from the controller 20 a time (detection timing) at which an unknown protein solution has been dropped on the sensor 1 (step S21). In step S21, the computation circuit 30 does not necessarily have to acquire from the controller 20 a time at which an unknown protein solution has been dropped on the sensor 1 and may receive the input of dropping timing from a user.

The computation circuit 30 causes the state space model analysis portion 31 to perform analysis by using results of the parameters a and b of the response model qₜ determined in the learning phase for the above state space model (step S22). In the method of using results of the parameters a and b of the response model qₜ determined in the learning phase for the state space model, a representative point such as a mean value or a median value, for example, may be used or the parameter of distribution such as normal distribution, for example, may be used instead. The computation circuit 30 may perform analysis by using all pieces of data used in the estimation of the parameters a and b of the response model qₜ for the state space model. The computation circuit 30 calculates the protein solution concentration (detection target concentration) c from the response model qₜ (step S23).

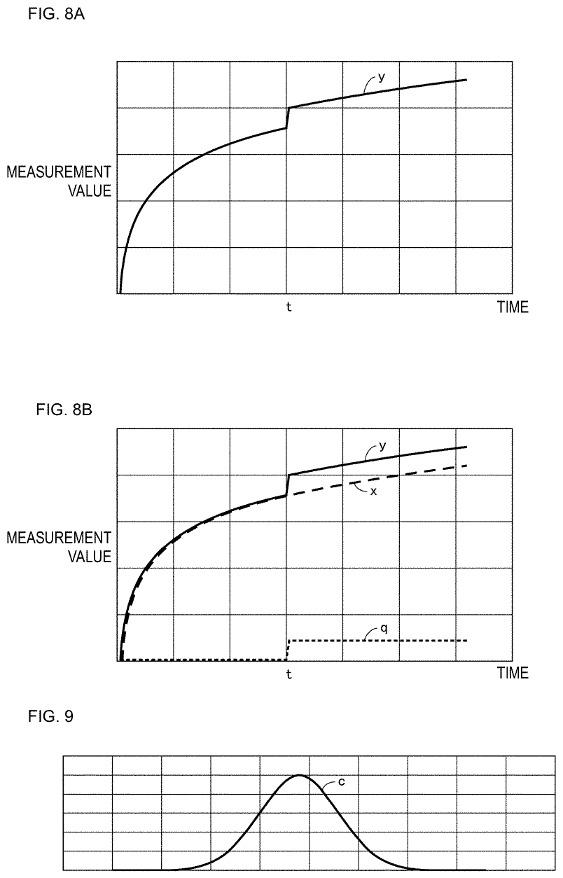

Next, a concrete example of the prediction phase will be described. FIGS. 8A and 8B are graphs representing the change in measurement value in the prediction phase in the first preferred embodiment. FIG. 8A illustrates the change in the signal (current value measured by the measurement circuit 10) y from the sensor 1. The vertical axis represents measurement value and the horizontal axis represents time. FIG. 8B illustrates the changes in the signal (current value measured by the measurement circuit 10) y from the sensor 1, the variation component (drift component) x of the sensor 1, and the response component (response model) q of the sensor 1. The vertical axis represents measurement value and the horizontal axis represents time.

For example, the measurement values in FIGS. 8A and 8B are obtained by continuously monitoring a drain current in a state where a predetermined voltage is applied to the gate electrode and the drain electrode of the sensor 1 in a graphene FET. FIGS. 8A and 8B illustrate changes in measurement value when a protein solution of unknown concentration is dropped in the buffer solution 1b at a time t.

As illustrated in FIG. 8A, the signal (current value measured by the measurement circuit 10) y from the sensor 1 represented by a solid line nonlinearly changes even before a protein solution of unknown concentration is dropped, and a variation component (drift component) is superimposed on the signal. When a protein solution of unknown concentration is dropped at the time t, the measurement value of the signal y from the sensor 1 rapidly increases.

The computation circuit 30 can separate the signal y from the sensor 1 into the variation component (drift component) x of the sensor 1 and the response component (response model) q of the sensor 1 as illustrated in FIG. 8B by performing analysis by using results of the parameters a and b of the response model qₜ determined in the learning phase for the above state space model in step S22. In an analysis performed using a state space model, the variation component x of the sensor 1 and the response component q of the sensor 1 can be subjected to distribution estimation rather than point estimation. FIG. 8B illustrates the mean value of the variation components x of the sensor 1 obtained by distribution estimation and the mean value of the response components q of the sensor 1 obtained by distribution estimation. By analyzing the variation component of the sensor 1 using a state space model, the variation component of the sensor 1 can be quantitatively determined separately from the response component q of the sensor 1 even if a function for the variation component of the sensor 1 is not known in advance.

The computation circuit 30 causes the simulation portion to calculate the protein solution concentration (detection target concentration) c from the response model qₜ using the MCMC method. FIG. 9 is a diagram illustrating the protein solution concentration (detection target concentration) c calculated in the prediction phase in the first preferred embodiment. FIG. 9 illustrates the distribution of the calculated protein solution concentration (detection target concentration) c. Since the protein solution concentration (detection target concentration) c can be calculated, an unknown protein solution can be evaluated. In FIG. 9, the horizontal axis represents the value of the concentration of a protein solution and the vertical axis represents frequency.

In the state space model analyzed as illustrated in FIGS. 4A to 6B, 8A, 8B, and 9, a state equation is xₜ=Gxₜ₋₁+wₜ, an observation equation is yₜ=Fxₜ+qₜ+vₜ, only the response model qₜ is a nonlinear function, and the other terms have a linear or Gaussian distribution. Here, G represents, for example, a matrix with two rows and two columns, and F represents, for example, a matrix with one row and two columns. Each element in the respective matrices is a constant. However, the state space model is not limited thereto. The state equation and the observation equation may include a nonlinear function in addition to the response model qₜ.

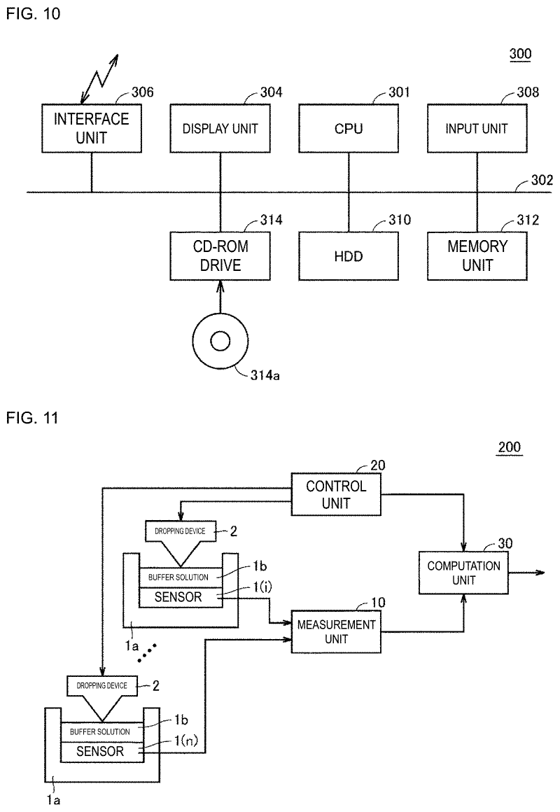

The controller 20 and the computation circuit 30, which have been described above, can be, for example, a computer 300. FIG. 10 is a block diagram illustrating the configuration of the computer 300 according to the first preferred embodiment. The computer 300 includes a CPU 301 that executes various programs including an operating system (OS), a memory 312 that temporarily stores data required for the execution of a program in the CPU 301, and a hard disk drive (HDD) 310 that stores a program executed by the CPU 301 in a non-volatile manner. The hard disk drive 310 stores in advance, for example, programs for the achievement of analysis of a state space model in the learning phase and the prediction phase. Such a program is read from a storage medium such as a CD-ROM (compact disc-read-only memory) 314a by, for example, a CD-ROM drive 314.

The CPU 301 receives an instruction from a user via an input device 308 including a keyboard and a mouse and outputs, for example, a result of analysis performed by the execution of a program to a display 304. The respective portions are connected to each other via a bus 302. An interface 306 is to be connected to an external device such as, for example, the measurement circuit 10 and the dropping device 2. The connection between the computer 300 and an external device may be established in a wired or wireless manner.

As described above, the detector 100 according to the present preferred embodiment detects a target using the sensor 1 and includes the measurement circuit 10 that measures a signal from the sensor 1 and the computation circuit 30 that separates a signal measured by the measurement circuit 10 into the variation component and the response component of the sensor 1. The computation circuit 30 includes the state space model analysis portion 31 that performs analysis using a state space model including a state equation specified by the time-series information of the variation component of the sensor 1 and an observation equation specified by separation between the variation component of the sensor 1 and the response component of the sensor and the parameter determination portion 33 that determines parameters included in the state space model used by the state space model analysis portion 31. The computation circuit 30 obtains a target corresponding to the response component using the parameters (the parameters a and b of the response model qₜ) determined by the parameter determination portion 33.

Since the computation circuit 30 in the detector 100 according to the present preferred embodiment performs analysis using a state space model including a state equation specified by the time-series information of the variation component of the sensor 1 and an observation equation specified by the separation between the variation component of the sensor 1 and the response component of the sensor 1, a target (e.g., the protein solution concentration c) can be accurately detected without adding another piece of hardware and waiting until the target goes into a steady state.

The detector 100 according to the present preferred embodiment models the variation component of the sensor 1 and the response component of the sensor 1 separately from each other in the state space model and defines the variation component of the sensor 1, which cannot be subjected to strict formulation, as a state equation specified by time-series information. As a result, the characteristics of the sensor 1 represented by a complex response model that is a nonlinear function can be estimated.

In the detector 100 according to the present preferred embodiment, a signal from the sensor 1 is handled as a typical state space model. Accordingly, the parameters of distributions of observation noise and system noise, which need to be determined in advance in Kalman filters, can be collectively analyzed along with the parameters a and b of the response model qₜ. As a result, the accuracy of estimating the parameters a and b of the response model qₜ can be improved. Since the detector 100 according to the present preferred embodiment uses a state space model, the scheme of the Bayes estimation, an appropriate model using an information criterion such as AIC, BIC, WAIC, or WBIC can be selected. The detector 100 according to the present preferred embodiment may propose a plurality of conceivable state space models in advance, provide time-series information for the respective state space models for comparison between information criteria, and select the most appropriate state space model.

The detector 100 according to the present preferred embodiment may further include the controller 20 that controls a computation phase in the computation circuit 30. When the controller 20 controls a computation phase in the computation circuit 30 to the learning phase (first computation phase), the parameter determination portion 33 applies a known target and response information obtained from the known target to the state space model and determines the parameters a and b of the response model qₜ representing a relationship between the target and a response component. When the controller 20 controls a computation phase in the computation circuit 30 to the prediction phase (second computation phase), the state space model analysis portion 31 separates a signal measured by the measurement circuit 10 into the variation component and the response component of the sensor 1 and obtains a target (e.g., the protein solution concentration c) corresponding to the response component using the parameters a and b of the response model qₜ determined in the learning phase. With this configuration, the detector 100 according to the present preferred embodiment can switch between the determination of the parameters a and b of the response model qₜ and the calculation of the protein solution concentration (detection target concentration) c based on a computation phase.

The observation equation may be the response model qₜ in which the response component of the sensor 1 is nonlinear. With this configuration, the detector 100 according to the present preferred embodiment can express the relationship between a target and a response component using the response model qₜ in an optimal manner.

The computation circuit 30 may further include the simulation portion 32 that performs mathematical calculation of the state space model by simulation. The simulation portion 32 calculates the parameters a and b of the response model qₜ by simulation in the learning phase (first computation phase) and obtains from the response model qₜ a target (e.g., the protein solution concentration c) corresponding to a response component by simulation in the prediction phase (second computation phase). With this configuration, the detector 100 according to the present preferred embodiment can estimate the parameters a and b and obtain a target even when the response model qₜ is nonlinear. The simulation portion 32 may perform mathematical calculation of the state space model using the Markov chain Monte Carlo method, for example.

A non-limiting example of a detection method of the detector 100 according to the present preferred embodiment includes the step (step S10 to S13) of, when the controller 20 controls a computation phase in the computation circuit 30 to the learning phase (first computation phase), causing the parameter determination portion 33 to apply a known target and response information obtained from the known target to the state space model and determine the parameters a and b of the response model qₜ representing a relationship between a target and a response component and the step (step S20 to S23) of, when the controller 20 controls a computation phase in the computation circuit 30 to the prediction phase (second computation phase), causing the state space model analysis portion 31 to separate a signal measured by the measurement circuit 10 into the variation component and the response component of the sensor 1 and obtain a target (e.g., the protein solution concentration c) corresponding to the response component using the parameters a and b of the response model qₜ determined in the learning phase.

Since the computation circuit 30 in the detector 100 according to the present preferred embodiment performs analysis using a state space model including a state equation specified by the time-series information of the variation component of the sensor 1 and an observation equation specified by the separation between the variation component of the sensor 1 and the response component of the sensor 1, the detection method of the detector 100 according to the present preferred embodiment enables a target (e.g., the protein solution concentration c) to be accurately detected without adding another piece of hardware and waiting until the target goes into a steady state.

A program that is executed by the computation circuit 30 in the detector 100 according to the present preferred embodiment executes the step (step S10 to S13) of, when the controller 20 controls a computation phase in the computation circuit 30 to the learning phase (first computation phase), causing the parameter determination portion 33 to apply a known target and response information obtained from the known target to the state space model and determine the parameters a and b of the response model qₜ representing a relationship between a target and a response component and the step (step S20 to S23) of, when the controller 20 controls a computation phase in the computation circuit 30 to the prediction phase (second computation phase), causing the state space model analysis portion 31 to separate a signal measured by the measurement circuit 10 into the variation component and the response component of the sensor 1 and obtain a target (e.g., the a protein solution concentration c) corresponding to the response component using the parameters a and b of the response model qₜ determined in the learning phase.

Since the computation circuit 30 in the detector 100 according to the present preferred embodiment performs analysis using a state space model including a state equation specified by the time-series information of the variation component of the sensor 1 and an observation equation specified by the separation between the variation component of the sensor 1 and the response component of the sensor 1, a program executed by the computation circuit 30 enables a target (e.g., the protein solution concentration c) to be accurately detected without adding another piece of hardware and waiting until the target goes into a steady state.

### Second Preferred Embodiment

In the first preferred embodiment, the detector 100 illustrated in FIG. 1 in which the single sensor 1 is connected to the measurement circuit 10 has been described. In a second preferred embodiment of the present invention, a detector in which a plurality of sensors are connected to a measurement circuit will be described. FIG. 11 is a schematic diagram illustrating the configuration of a detector 200 according to the second preferred embodiment. The configurations of the detector 200 in FIG. 11 that are the same or substantially the same as those of the detector 100 in FIG. 1 will be denoted by the same reference numerals, and a detailed description thereof will not be repeated.

In the detector 200 illustrated in FIG. 11, the sensor 1 is an array sensor including a plurality of sensor elements. A single sensor element in an array sensor corresponds to the sensor 1 illustrated in FIG. 1, and each sensor element is represented by 1(i) (i=1 to n). The configuration of an array sensor is not limited to a configuration in which sensor elements are provided in a matrix and may be a configuration in which a plurality of independent sensors are provided. FIG. 11 illustrates the configuration of an array sensor in which the multiple sensors 1 illustrated in FIG. 1 are provided.

Since the array sensor illustrated in FIG. 11 has the configuration in which the multiple sensors 1 illustrated in FIG. 1 are provided, the dropping device 2 is provided for each of the sensors 1. However, the dropping device 2 does not necessarily have to be provided for each of the sensors 1, and a configuration may be provided in which the single dropping device 2 is provided for the multiple sensors 1.

The sensors 1(i) included in the array sensor are connected to the measurement circuit 10. Signals from the respective sensors 1(i) are analyzed by the computation circuit 30 using state space models corresponding to the respective sensors 1(i). The computation circuit 30 performs computation to separate a signal measured by each of the sensors 1(i) (sensor elements) into the variation component and the response component of the sensor 1 as described in the first preferred embodiment. The analysis may be performed using a single state space model associated with the sensors 1(i) or independent state space models associated with the respective sensors 1(i).

Next, a learning phase (first computation phase) in the computation phase of the computation circuit 30 will be described. The learning phase is a computation phase in which the parameters a and b of the response model qₜ of each of the sensors 1(i) are determined. Specifically, in the learning phase, the parameters a and b of the response model qₜ are determined for each of the sensors 1(i) based on a signal that is output from the sensor 1(i) after a protein solution of known concentration has been dropped on the sensor 1(i).

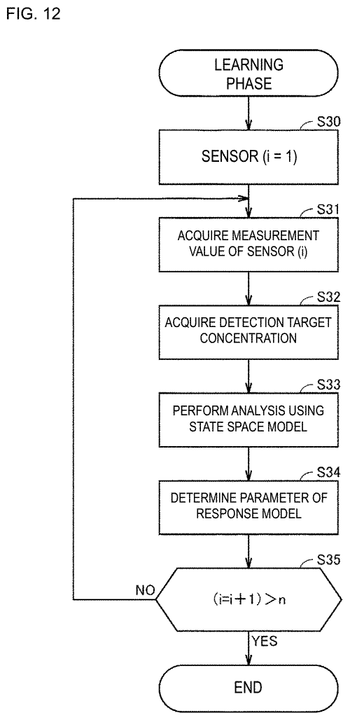

FIG. 12 is a flowchart of the learning phase in the second preferred embodiment. In the flowchart illustrated in FIG. 12, the respective sensors 1(i) are independently analyzed. First, the computation circuit 30 specifies the sensor 1(i) upon which computation is to be performed (step S30). Subsequently, the computation circuit 30 acquires from the measurement circuit 10 a measurement value (current value) measured by the sensor 1(i) (step S31). Subsequently, the computation circuit 30 acquires from the controller 20 a known protein solution concentration (detection target concentration) (step S32). In step S32, the computation circuit 30 does not necessarily have to acquire a known protein solution concentration from the controller 20 and may receive the input of a known protein solution concentration from a user.

The computation circuit 30 causes the state space model analysis portion 31 to perform analysis using the state space model described in the first preferred embodiment (step S33). The computation circuit 30 causes the simulation portion 32 to perform mathematical calculation by simulation upon the state space model described in the first preferred embodiment to determine the parameters a and b of the response model qₜ of the sensor 1(i) (step S34).

Subsequently, the computation circuit 30 changes the sensor 1(i) upon which computation is to be performed to the sensor 1(i=i+1) and determines whether the number (i=i+1) of the sensor 1 is greater than n (step S35). When the number (i=i+1) of the sensor 1 is not greater than n ((i=i+1)>n is not established) (NO in step S35), the computation circuit 30 performs the process from steps S31 to S34 upon a signal from the sensor 1(i=i+1). When the number (i=i+1) of the sensor 1 is greater than n ((i=i+1)>n is established) (YES in step S35), the computation circuit 30 determines that computation has been performed upon all of the sensors 1, the sensor 1(1) to the sensor 1(n), and ends the process. Computation performed upon each of the sensors 1(i) is the same or substantially the same as that performed upon the sensor 1 described in the first preferred embodiment, and the detailed description thereof will not be repeated.

Next, a prediction phase (second computation phase) in the computation phase of the computation circuit 30 will be described. The prediction phase is a computation phase in which an unknown concentration of a protein solution is predicted using results of the parameters a and b of the response model qₜ of each of the sensors 1 determined in the learning phase. Specifically, in the prediction phase, a protein solution of unknown concentration is dropped on each of the sensors 1(i) and the concentration of the protein solution is obtained based on a signal from the sensor 1(i). The dropping of different protein solutions upon the respective sensors 1(i) enables many protein solution concentrations to be detected in a single piece of detection processing.

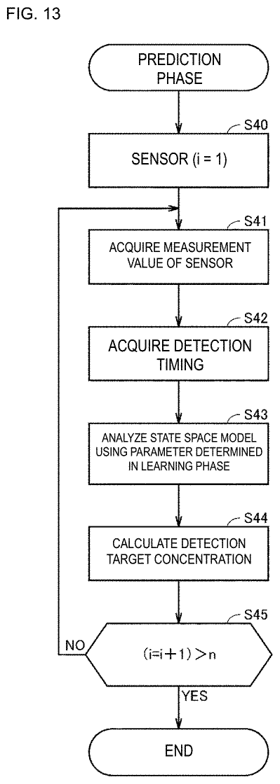

FIG. 13 is a flowchart of the prediction phase according to the second preferred embodiment. First, the computation circuit specifies the sensor 1(i) upon which computation is to be performed (step S40). Subsequently, the computation circuit 30 acquires from the measurement circuit 10 a measurement value (current value) measured by the sensor 1(i) (step S41). Subsequently, the computation circuit 30 acquires from the controller 20 a time (detection timing) at which an unknown protein solution has been dropped on the sensor 1(i) (step S42). In step S42, the computation circuit 30 does not necessarily have to acquire from the controller 20 a time at which an unknown protein solution has been dropped on the sensor 1(i) and may receive the input of dropping timing from a user.

The computation circuit 30 causes the state space model analysis portion 31 to perform analysis by using results of the parameters a and b of the response model qₜ of the sensor 1(i) determined in the learning phase for the state space model described in the first preferred embodiment (step S43). In the method of using the results of the parameters a and b of the response model qₜ of the sensor 1(i) determined in the learning phase for the state space model, a representative point such as a mean value or a median value, for example, may be used or the parameter of distribution such as normal distribution, for example, may be used instead. The computation circuit 30 may perform analysis by using all pieces of data used in the estimation of the parameters a and b of the response model qₜ of the sensor 1(i) for the state space model. The computation circuit 30 calculates the protein solution concentration (detection target concentration) c from the response model qₜ of the sensor 1(i) (step S44).

Subsequently, the computation circuit 30 changes the sensor 1(i) upon which computation is to be performed to the sensor 1(i=i+1) and determines whether the number of the sensor 1(i=i+1) is greater than n (step S45). When the number (i=i+1) of the sensor 1 is not greater than n ((i=i+1)>n is not established) (NO in step S45), the computation circuit 30 performs the process from steps S41 to S44 upon a signal from the sensor 1(i=i+1). When the number (i=i+1) of the sensor 1 is greater than n ((i=i+1)>n is established) (YES in step S45), the computation circuit 30 determines that computation has been performed upon all of the sensors 1, the sensor 1(1) to the sensor 1(n), and ends the process.

Computation performed upon each of the sensors 1(i) is the same or substantially the same as that performed upon the sensor 1 described in the first preferred embodiment, and the detailed description thereof will not be repeated. The state space model analysis portion 31 may provide different prior distributions for the parameters a and b of the response models qₜ of the respective sensors 1(i) (respective sensor elements). For example, when the values of the parameters a and b of the response model qₜ have tendencies in accordance with the location of the sensor 1(i), the state space model analysis portion 31 may receive a prior distribution reflecting the tendencies and perform computation of the learning phase (first computation phase). A prior distribution to be provided for the state space model analysis portion 31 may be determined in advance for each of the sensors 1(i) or estimated for each of the sensors 1(i) using the hierarchical Bayesian model.

The detector 200 may drop different types of protein solutions on the respective sensors 1(i) and obtain the protein solution concentrations (detection target concentrations) c in the respective sensors 1(i). Alternatively, the detector 200 may drop the same protein solution on the respective sensors 1(i) and obtain the single protein solution concentration (detection target concentration) c in the sensors 1(i). In this case, the detector 200 may obtain the protein solution concentrations (detection target concentrations) c in the respective sensors 1(i) and calculate the mean value of them. Alternatively, the detector 200 performs analysis using a single state space model associated with the sensors 1(i) and obtain the single protein solution concentration (detection target concentration) c in the sensors 1(i).

In the detector 200 according to the second preferred embodiment, the concentration of a protein solution can be detected using the multiple sensors 1(i). Accordingly, the parameter determination portion 33 may determine whether the parameters a and b of the response model qₜ of each of the sensors 1(i) determined in the learning phase (first computation phase) meets a predetermined criterion, and the state space model analysis portion 31 does not necessarily have to perform computation for the sensor 1(i) having the parameters that do not meet the predetermined criterion in the prediction phase (second computation phase). The predetermined criterion needs to be determined in advance. Examples of the method of determining whether the parameters meet a criterion include a method of substituting the representative values (e.g., mean values, median values, or variances) of the parameters a and b of the response model qₜ estimated as distributions as illustrated in FIGS. 6A and 6B into a distribution prepared in advance and determining whether the likelihoods (or log likelihoods) thereof meet the predetermined criterion. As the method of determining whether the parameters meet a criterion, a method may include obtaining a degree of similarity between a parameter distribution using an indicator such as KL-divergence, for example, and a distribution prepared in advance (e.g., the reciprocal of KL-divergence) and determining whether the degree of similarity meets a criterion.

As described above, in the detector 200 according to the second preferred embodiment, the sensor 1 is an array sensor including a plurality of sensor elements. The computation circuit 30 performs computation to separate a signal measured by each of the sensors 1(i) (sensor elements) into the variation component and the response component of the sensor 1(i).

Since the detector 200 according to the present preferred embodiment determines the parameters a and b of the response model qₜ of each of the sensors 1(i) and obtains a target using the parameters a and b, the detector 200 can accurately detect the target regardless of the variation in characteristics of sensor elements.

The state space model analysis portion 31 may provide different prior distributions for the parameters a and b of the response models qₜ of the respective sensors 1(i) (respective sensor elements). The detector 200 can therefore reflect an individual difference in each of the sensors 1(i) and estimate parameters without uniformity and with flexibility.

The parameter determination portion 33 may determine whether the parameters a and b of the response model qₜ of each of the sensors 1(i) determined in the learning phase (first computation phase) meets a predetermined criterion, and the state space model analysis portion 31 does not necessarily have to perform computation for the sensor 1(i) having the parameters that do not meet the predetermined criterion in the prediction phase (second computation phase). Since the detector 200 can remove a result of the sensor 1(i) that cannot be used for the detection of a target, the detector 200 can accurately detect a target.

### Other Modifications

(1) In the above-described preferred embodiments, the detectors 100 and 200 determine the parameters a and b of the response model qₜ in the learning phase (first computation phase) and obtain a target (e.g., the protein solution concentration c) using the determined parameters a and b of the response model qₜ in the prediction phase (second computation phase). The detectors 100 and 200 may perform the learning phase (first computation phase) and the prediction phase (second computation phase) each time detection processing is performed, or may perform the prediction phase (second computation phase) a plurality of times after performing the learning phase (first computation phase) one time. For example, the detectors 100 and 200 may perform the learning phase (first computation phase) one time at the time of startup and then perform only the prediction phase (second computation phase) to obtain a target (e.g., the protein solution concentration c). Different detectors may be used for the learning phase (first computation phase) and the prediction phase (second computation phase).

(2) In the detector 200 according to the second preferred embodiment, the concentrations of different types of protein solutions may be detected, and it may be determined whether the concentrations meet a criterion concentration in the respective sensors 1(i). For example, when the detector 200 is used for the detection of a cancer marker, a specimen including a cancer marker of concentration higher than or equal to a criterion concentration can be automatically determined from many specimens.

(3) In the detector 200 according to the second preferred embodiment, the different response models qₜ may be set for the respective sensors 1(i) (respective sensor elements) in the state space models. As a result, the detector 200 can analyze the state space model based on the characteristics of each of the sensors 1(i).

(4) The various processes described above are performed by the CPU 301 in the computer 300, for example, but do not necessarily have to be performed by the CPU 301. For example, these various functions may be performed by at least one semiconductor integrated circuit such as a processor, at least one ASIC (application-specific integrated circuit), at least one DSP (digital signal processor), at least one FPGA (field programmable gate array), and/or another circuit having a computation function.

These circuits can perform the above various processes by reading one or more commands from at least one tangible readable medium.

Such a medium is, for example, an optional type of memory such as a magnetic medium (e.g., hard disk), an optical medium (e.g., compact disc (CD) or DVD), a volatile memory, or a nonvolatile memory, but does not necessarily have to be a memory.

Examples of a volatile memory include a DRAM (dynamic random access memory) and an SRAM (static random access memory). Examples of a nonvolatile memory include a ROM and an NVRAM.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A detector for detecting a target using a sensor, the detector comprising:
a measurement circuit to measure a signal from the sensor; and
a computation circuit to separate a signal measured by the measurement circuit into a variation component of the sensor and a response component of the sensor; wherein
the computation circuit includes:
a state space model analysis portion to perform analysis using a state space model including a state equation specified by time-series information of a variation component of the sensor and an observation equation specified by separation between a variation component of the sensor and a response component of the sensor; and
a parameter determination portion to determine a parameter included in the state space model used by the state space model analysis portion; and
the computation circuit is configured to obtain a target corresponding to a response component using a parameter determined by the parameter determination portion.

2. The detector according to claim 1, further comprising:
a controller to control a computation phase in the computation circuit; wherein
when the controller is configured or programmed to control a computation phase in the computation circuit to a first computation phase, the parameter determination portion applies a known target and response information obtained from the known target to the state space model and determines a parameter of a response model representing a relationship between a target and a response component; and
when the controller is configured or programmed to control a computation phase in the computation circuit to a second computation phase, the state space model analysis portion separates a signal measured by the measurement circuit into a variation component of the sensor and a response component of the sensor and obtains the target corresponding to a response component using the parameter of the response model determined in the first computation phase.

3. The detector according to claim 2, wherein the observation equation is the response model in which a response component of the sensor is nonlinear.

4. The detector according to claim 3, wherein
the computation circuit further includes a simulation portion to perform mathematical calculation of the state space model by simulation; and
the simulation portion calculates a parameter of the response model by simulation in the first computation phase and obtains from the response model a target corresponding to a response component by simulation in the second computation phase.

5. The detector according to claim 4, wherein the simulation portion performs mathematical calculation of the state space model using a Markov chain Monte Carlo method.

6. The detector according to claim 2, wherein
the sensor is an array sensor including a plurality of sensor elements; and
the computation circuit performs computation to separate a signal measured by each of the plurality of sensor elements into a variation component of the sensor and a response component of the sensor.

7. The detector according to claim 6, wherein the state space model analysis portion provides different prior distributions for parameters of the response models of the respective sensor elements.

8. The detector according to claim 6, wherein
the parameter determination portion determines whether parameters of the response models of the respective sensor elements determined in the first computation phase meet a predetermined criterion; and
the state space model analysis portion does not perform computation for the sensor element with a parameter that does not meet the predetermined criterion in the second computation phase.

9. A detection method of a detector that detects a target using a sensor and that includes a measurement circuit to measure a signal from the sensor, a computation circuit to separate a signal measured by the measurement circuit into a variation component of the sensor and a response component of the sensor, and a controller to control a computation phase in the computation circuit, the computation circuit including a state space model analysis portion to perform analysis using a state space model including a state equation specified by time-series information of a variation component of the sensor and an observation equation specified by separation between a variation component of the sensor and a response component of the sensor, and a parameter determination portion to determine a parameter included in the state space model used by the state space model analysis portion, the detection method comprising:
causing the parameter determination portion, when the controller controls a computation phase in the computation circuit to a first computation phase, to apply a known target and response information obtained from the known target to the state space model and determine a parameter of a response model representing a relationship between a target and a response component; and
causing the state space model analysis portion, when the controller controls a computation phase in the computation circuit to a second computation phase, to separate a signal measured by the measurement circuit into a variation component of the sensor and a response component of the sensor and obtain the target corresponding to a response component using the parameter of the response model determined in the first computation phase.

10. A non-transitory computer readable medium executable by a computation circuit in a detector that detects a target using a sensor and that includes a measurement circuit to measure a signal from the sensor, the computation circuit being able to execute a program to separate a signal measured by the measurement circuit into a variation component of the sensor and a response component of the sensor, and a controller to control a computation phase in the computation circuit, the computation circuit including a state space model analysis portion to perform analysis using a state space model including a state equation specified by time-series information of a variation component of the sensor and an observation equation specified by separation between a variation component of the sensor and a response component of the sensor, and a parameter determination portion configured to determine a parameter included in the state space model used by the state space model analysis portion, the program causing the computation circuit to:
cause the parameter determination portion, when the controller controls a computation phase in the computation circuit to a first computation phase, to apply a known target and response information obtained from the known target to the state space model and determine a parameter of a response model representing a relationship between a target and a response component; and
cause the state space model analysis portion, when the controller controls a computation phase in the computation circuit to a second computation phase, to separate a signal measured by the measurement circuit into a variation component of the sensor and a response component of the sensor and obtain the target corresponding to a response component using the parameter of the response model determined in the first computation phase.

11. The detector according to claim 1, wherein the sensor is a graphene FET sensor.

12. The detector according to claim 11, wherein the graphene FET sensor is provided in a casing and includes an upper surface filled with a buffer solution.

13. The detector according to claim 12, wherein the buffer solution includes phosphate buffered salts.

14. The detector according to claim 12, further comprising a dropping device to drop a protein solution in the buffer solution.

15. The detection method according to claim 9, wherein the sensor is a graphene FET sensor.

16. The detection method according to claim 15, wherein the graphene FET sensor is provided in a casing and includes an upper surface filled with a buffer solution.

17. The detection method according to claim 16, wherein the buffer solution includes phosphate buffered salts.

18. The detection method according to claim 16, further comprising a dropping device to drop a protein solution in the buffer solution.
